(19) United States
(12) Patent Application Publication
(10) Pub. No.: US 2025/0273093 A1
(43) Pub. Date: Aug. 28, 2025

(54) ARRANGEMENT APPLIED TO A SIMULATOR FOR OPHTHALMOLOGICAL SURGERY TRAINING AND ARTIFICIAL CATARACT

(71) Applicant: CÂNDIDO ALVES E SILVA, TAUANNI, Palmas (BR)

(72) Inventor: Tauanni CÂNDIDO ALVES E SILVA, Palmas (BR)

(21) Appl. No.: 18/859,373

(22) PCT Filed: Apr. 28, 2023
(86) PCT No.: PCT/BR2023/050133

§ 371 Date: Oct. 23, 2024

(30) Foreign Application Priority Data
Apr. 30, 2022 (BR) 2020220084063

(51) Int. Cl. G09B 23/34; G09B 23/32

(52) U.S. Cl. CPC G09B 23/34; G09B 23/32

(57) ABSTRACT

An arrangement applied to a simulator for surgical training in ophthalmology that is anatomically similar to the human eye, and has the purpose of training and qualification in ophthalmological surgical processes. An artificial cataract for use in simulators for surgical training in ophthalmology.

### FIELD OF APPLICATION

The present patent application aims at creating a synthetic model for surgical training in ophthalmology, for use by ophthalmologists during training and after their specialization, with a view to providing better surgical training and thus reducing the number of intra- and post-operative complications arising from the lack of practical experience.

### BACKGROUNDS

Current training possibilities for cataract surgery, glaucoma surgery and corneal transplantation mainly include in vivo training, that is, learning the surgical practice directly in the human eye, which is not desirable, given the risk of potentially existing complications, which can even cause permanent blindness. Another current practice is in vitro training, using animal eyes, a practice that limits learning because they do not have anatomical characteristics similar to the human eye, in addition to requiring complicated transportation and storage logistics, since most slaughtered animals have not yet developed cataracts and, after the second day, even when stored in a cool environment, the eyes already show signs of corneal opacification, compromising the performance of surgical training. Furthermore, the use of animal eyes fosters the existing bioethical conflict regarding animal experimentation.

In view of the disclosure above, the development of new technologies to train medical surgical skills is necessary and of great value in the current context, in which cataracts are the second cause of reversible blindness in the world. Therefore, trained surgeons are required to reverse this statistical situation.

Despite the use of existing training models, some drawbacks may be attributed thereto, such as, for example: the cornea being made of rigid material, which prevents adequate training of surgical incisions in the cornea; anterior and posterior chambers of the eye with anatomy and/or dimensions different from the human eye; the need for different models for training surgical steps existing in the continuum of the same surgery, thus representing, in a way that is not very similar to, the reality existing in the intraoperative in vivo; cataracts made of non-soluble synthetic material, which potentially obstructs the and tip causes damage to the phacoemulsification device used in the surgeries. Another problem with existing devices is that they do not allow for a close reproduction of reality regarding the behavior of collapse of some ocular structures when faced with a sudden reduction in pressure in the anterior chamber. This behavior is called “surge” and is perceived intraoperatively by the surgeon through the “subtle shrinkage/collapse” of some ocular structures. This step is essential for surgical training because the sudden pressure oscillation in the anterior chamber of the eye causes significant damage to the patient's corneal endothelium and must be promptly identified and corrected by the surgeon. Another difficulty with current models is representing ocular mobility, the surgical steps of hydrodissection, cataract rotation, rupture of the posterior capsule, capsulorhexis of the posterior capsule, in addition to corneal transplants.

In consonance with the disclosure above, document U.S. Pat. No. 6,589,057 B1 “Incision trainer for ophthalmological surgery” offers the practice of corneal incisions as it is a device with features similar to those of a human cornea, but it does not have an apparatus that simulates other structures such as the cataract with its respective anterior and posterior capsules, necessary for practical training of phacoemulsification.

Document BR 20 2020 006584 5 “Disposição aplicada em câmara artificial para simulação de cirurgia de transplante córnea” (“Arrangement applied in artificial chamber for simulation of corneal transplant surgery”), as it is a structure that consists of an artificial chamber and has features similar to those of the human cornea and the anterior chamber, offers the practice of corneal transplantation, but does not have structures that represent the other formations of the eye; therefore, it does not allow training in combined corneal transplantation and phacoemulsification surgery, in addition to not simulating the ocular movement that may occur during the manipulation of ocular structures intraoperatively.

Document BR 20 2020 006529 2 “Disposição aplicada em kit de câmara artificial para simulação de cirurgia de catarata” (“Arrangement applied in artificial chamber kit for simulating cataract surgery”) addresses to a device that simulates the anterior chamber with features similar to the human cornea and cataract, enabling training in phacoemulsification surgery, but with important limitations, mainly regarding intraoperative surgical complications involving the posterior capsule and posterior capsulotomy training, as it does not have structures that simulate the posterior chamber or posterior capsule, in addition to not having ocular mobility.

Document U.S. Pat. No. 8,845,334 B1 “Apparatus for practicing ophthalmologic surgical techniques” addresses to a device that enables training in some important surgical steps such as capsulorhexis and phacoemulsification techniques, but does not enable training in corneal incisions, which is the initial step of the surgery.

In view of such problems and with the purpose of overcoming the same, the simulator for surgical training in ophthalmology, which is the object of the present application, was developed, which consists of providing a device for cataract surgery, glaucoma surgery and corneal transplant training, in order to contemplate the testing of all surgical steps involved in the various existing techniques of cataract and glaucoma surgery, in addition to corneal transplant training, to improve the realistic simulation of the intraoperative period, maximize learning and reduce learning costs, as well as intra- and post-operative complications resulting from the lack of training and practical experience.

The present invention has as a feature a rounded structure, with a convex anterior surface and a concave posterior surface, in a material composed of polysiloxane polymers, with dimensions, thickness, elasticity, mechanical strength, texture and malleability similar to that of the human cornea, enabling training of the various existing surgical incisions, in addition to maintaining the existence of an anterior chamber without continuous entry of air bubbles, thus simulating the intraoperative reality of cataract surgery. The anterior capsule produced has relevant characteristics such as low mechanical strength, low resistance to rupture and slight elasticity, which make it very similar to the anterior capsule present in human cataracts and which allows training of a very important surgical step called capsulorhexis. The posterior capsule existing in the present application allows recreating the important surgical steps of hydrodissection and mechanical rotation of the cataract within this capsule, as well as training of the control of intraoperative complications, such as, for example, rupture of the posterior capsule, also enabling the practice of the posterior capsulorhexis existing in congenital cataract surgery.

It also allows the visualization of the so-called “surge”, which is the collapse behavior of ocular structures in response to sudden pressure fluctuations in the anterior chamber as described above. The structure that simulates the iris, in the present application, consists of a polysiloxane sheet with high malleability and moderate elasticity, which represents important characteristics of the human iris and allows the surgeon to touch and handle the same during the surgical simulation, making the experience even closer to reality.

The present invention allows training using the same materials and equipment used in vivo in the surgical centers of each service, therefore not requiring separate material exclusively for training, since the glycerinated saponaceous compound used in the structure that simulates the cataract is water-soluble and does not damage surgical equipment such as the phacoemulsifier, which reduces training costs.

Furthermore, the present invention allows the simulation of the number of cataract and glaucoma surgeries and corneal transplants that are necessary for the ophthalmologist in training, since the structures that perform the cornea and encapsulated cataract, among others, can be removed and replaced, which also reduces costs and makes training more accessible.

### OBJECTIVES

The present invention aims at improving surgical simulator devices, with the purpose of making the created reality closer to the reality existing during in vivo surgery, as well as greater accessibility to surgical training by reducing the costs involved, which provides better training for ophthalmologists and consequently a lower rate of intra- and post-operative complications resulting from lack of practical experience.

### DESCRIPTION OF THE DRAWINGS

FIG. 1 illustrates the external part of the simulator in oblique view.

FIG. 2 illustrates the details of all the structures present in the simulator in exploded oblique view.

FIG. 3 illustrates the side view of all the structures that make up the simulator.

FIG. 4a illustrates the perspective view of an embodiment of a cataract.

FIG. 4b illustrates the side view of the cataract.

FIG. 4c illustrates the anterior view of the cataract.

FIG. 4d illustrates the posterior view of the cataract.

FIG. 5 illustrates the simplified external part of a second embodiment of a simulator in oblique view.

FIG. 5a illustrates a simplified cross-sectional view of the second embodiment of the simulator.

FIG. 5b illustrates an enlarged sectional view of the second embodiment of the simulator.

### DETAILED DESCRIPTION

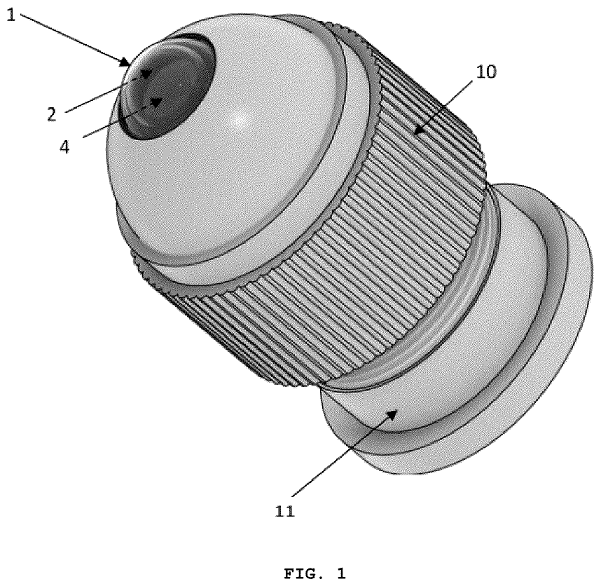

As can be seen in FIG. 1, this is a mobile eye model created in synthetic material, on a rigid base consisting of a hollow cylinder (11), preferably made of plastic, respecting the anatomical characteristics of the human eye relevant for cataract surgery and corneal transplantation. This model is specially adapted to simulate cataract surgery with intraocular lens implantation and corneal transplantation.

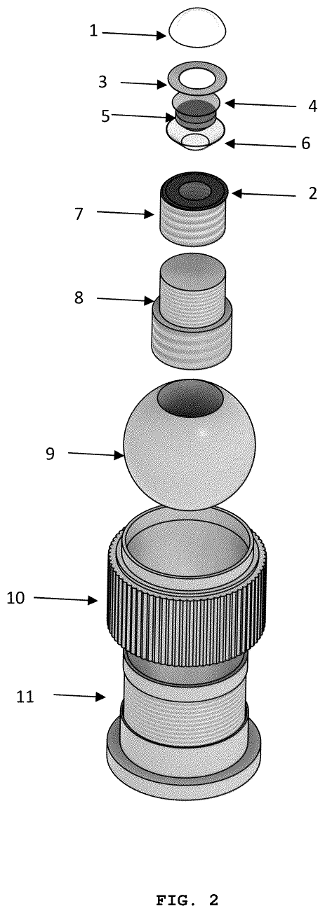

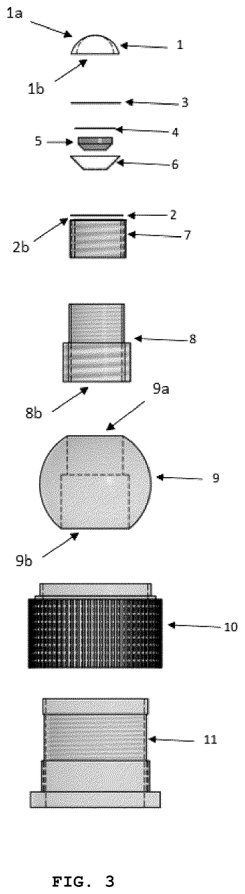

As can be seen in FIGS. 2 and 3, following a linear sequence from the anterior part to the posterior part, similar to the human organ, there is the synthetic cornea (1), which has a rounded shape, with a convex anterior transparent surface (1a) and a concave posterior transparent surface (1b), preferably composed of polysiloxane polymers, with dimensions, thickness, elasticity, mechanical strength, texture and malleability similar to the human cornea. Next, under the aforementioned transparent surface of the cornea (1), the device has an iris (2) consisting of a flat disk, preferably made of polysiloxane and adhesive vinyl, preferably blue in color, which tangentially extends along the entire internal perimeter of the cornea (1). Accordingly, the anterior chamber of the eye is formed by the space between the cornea (1) and the iris (2), with such a chamber having a spherical upper shape and a flat lower shape.

Just below the iris (2) and in contact with its posterior wall (2b), there is the encapsulated artificial cataract (5), which can be removable and replaceable, preferably made of glycerinated saponaceous material, surrounded anteriorly and posteriorly by two circular sheets, preferably of polyvinyl chloride, which characterize their respective anterior (4) and posterior (6) capsules, peripherally delimited by an acetate ring (3) that represents the zonule. The cataract (5) can be a biconvex or plano-convex lens. All the parts described above are supported in a non-definitive manner on a rigid cylindrical structure (7) that gives to the same support and allows the exchange of the items used after the end of the surgical training.

Next, coupled to the anterior chamber, there is a cylinder (8), preferably made of plastic material, which makes up the posterior chamber of the eye. This cylinder is fixed by its posterior portion (8b) to the larger posterior cut (9b) of a spherical structure (9) that represents the sclera, which also has a smaller anterior cut (9a), through which the cornea (1) is presented.

This spherical structure (9) has the potential for rotational movement, rests on the edges of a hollow plastic cylinder (11) and is sheltered at the top in its largest diameter by the internal edges of a second hollow cylinder (10), which partially covers the spherical structure (9) that represents the sclera, containing the same between the two cylinders (10 and 11) in order to allow the same to make rotational movements.

In a first embodiment of the present application, as illustrated in FIGS. 1 to 3, an arrangement applied to a simulator for surgical training in ophthalmology is disclosed, comprising at least one cornea (1), with a convex anterior surface (1a) and a concave posterior surface (1b), wherein, under the surface of the cornea (1), there is an iris (2) consisting of a flat disk that tangentially extends along the entire internal perimeter of the cornea (1); below the iris (2) and in contact with its posterior wall (2b), there is the cataract (5); the cataract (5) is surrounded anteriorly and posteriorly by two circular sheets that characterize their respective anterior (4) and posterior (6) capsules, peripherally delimited by a ring (3); below, there is a cylindrical structure (7); in sequence and in coupling, there is a cylinder (8) that is fixed by its posterior portion (8b) to the larger posterior cut (9b) of a spherical structure (9); the spherical structure (9) also has a smaller anterior cut (9a) through which the cornea (1) is presented; the spherical structure (9) rests on the edges of a hollow cylinder (11) and is superiorly sheltered in its largest diameter by the internal edges of a second hollow cylinder (10), which partially covers the spherical structure (9).

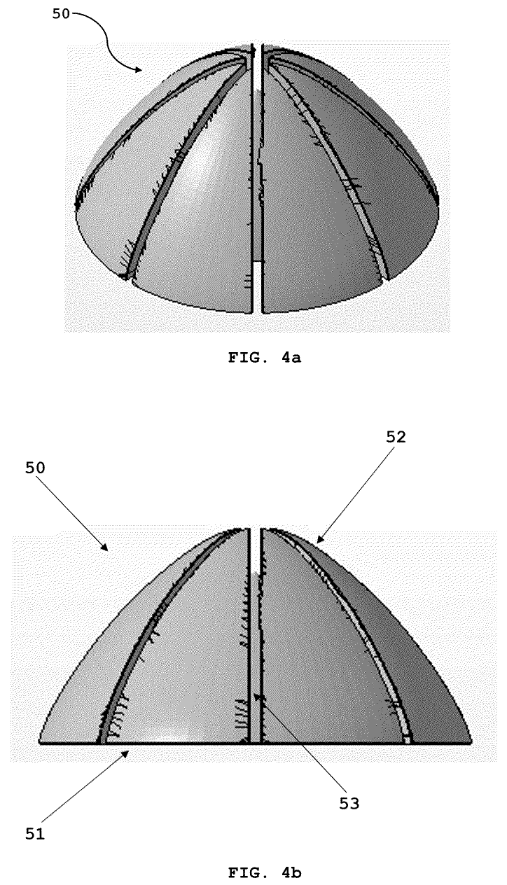

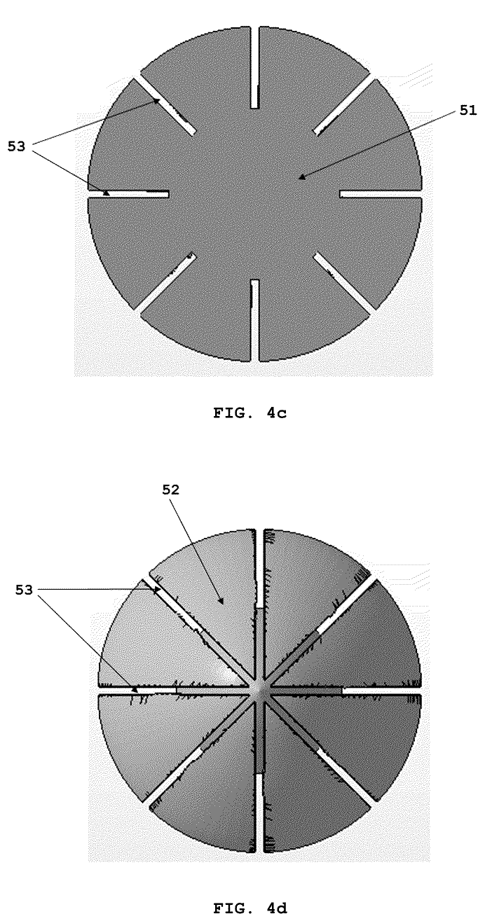

In a second embodiment of the present application, as illustrated in FIGS. 4a to 4d, a new configuration of an artificial cataract (50) for use in simulators for surgical training in ophthalmology is disclosed. Said cataract (50) may have a biconvex (not illustrated) or plano-convex (illustrated) lens shape.

Preferably, an anterior part (51) of the cataract (50) may have a plano or convex lens shape, while a posterior part (52) preferably comprises a convex lens shape. It will be appreciated by a technician skilled on the subject that the convexity of both the anterior part (51) and the posterior part (52) can be independently adjusted to simulate different surgical conditions.

Essentially, the novel configuration comprises a plurality of grooves (53) arranged evenly spaced around the cataract (50). The grooves (53) may extend from the anterior part (51) of the cataract (50) to meet at the convex posterior part (52) of the cataract (50), as illustrated in FIG. 4d. Optionally, the grooves (53) may extend only partially inwardly (not illustrated) the upper part (51), that is, so that the grooves (53) do not extend all the way to the posterior part (52) of the cataract (50).

The grooves (53) of the novel artificial cataract (50) configuration are advantageous in that they provide a more faithful simulation of cleavage planes. The cleavage planes are used during surgery to perform the breaking/rupturing and suction of the cataract parts. It will be appreciated that, depending on the number, thickness and/or depth of the grooves (53) in the artificial cataract (50), it is possible to alter the simulated force required to break the cleavage planes of the cataract (50). Accordingly, the proposed artificial cataract (50) advantageously allows to simulate various degrees of degeneration of an eye for surgical training in ophthalmology.

It will further be appreciated that the number, thickness and/or depth of the grooves (53) in the cataract (50) can be varied according to the dimensions of the cataract (50) that is desired to be simulated.

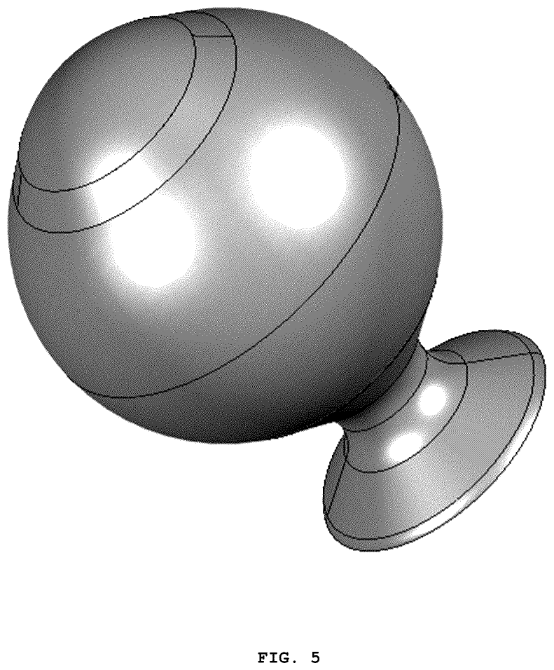

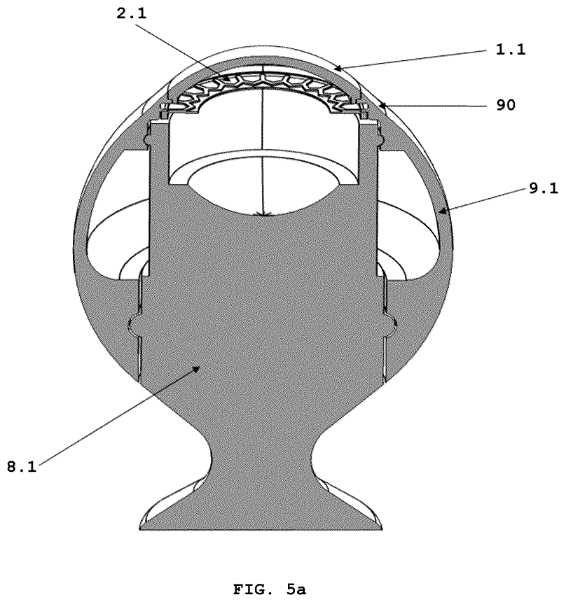

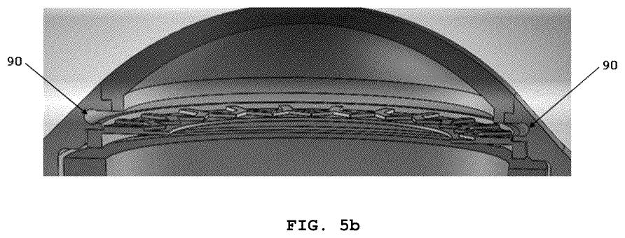

In a third embodiment of the present application, as illustrated in FIGS. 5 to 5b, an arrangement applied to a simulator for surgical training in ophthalmology is disclosed. It will be appreciated that the third embodiment comprises adaptations in relation to some of the elements of the first embodiment.

For illustrative purposes, the third embodiment comprises at least a cornea (1.1), an iris (2.1), a sclera (9.1), a cylinder (8.1) and an internal channel (90).

With respect to the first embodiment, the third embodiment differs essentially in that it comprises an internal channel (90) surrounding an internal part in the simulator, as illustrated in FIG. 5a. It will be appreciated that the internal channel (90) represents Schlemm's canal in the simulated eye.

The internal channel (90) may be positioned in the sclera (9.1), at a location close to the cornea (1.1) and the iris (2.1), as illustrated in FIG. 5b. Alternatively (not illustrated), the internal channel (90) may be positioned in the cornea (1.1), at a location close to the sclera (9.1) and the iris (2.1). Further alternatively (not illustrated), the internal channel (90) may be positioned at a junction site between the cornea (1.1) and the sclera (9.1).

It will further be appreciated that the internal channel (90), in the assembled configuration of the simulator for surgical training in ophthalmology, is a closed channel, with a substantially tubular shape.

The possibility of simulating Schlemm's canal by means of the internal channel (90) is advantageous for cases of simulating glaucoma surgery. In these cases, there is generally an obstruction in Schlemm's canal, which leads to high intraocular pressure, which can cause the ocular disease glaucoma.

Those skilled in the art will value the knowledge presented herein and will be able to reproduce the invention in the embodiments presented and in other variants, encompassed by the scope of the attached claims.

1. AN ARRANGEMENT APPLIED TO A SIMULATOR FOR SURGICAL TRAINING IN OPHTHALMOLOGY, comprising a cornea having a convex anterior surface and a concave posterior surface; below a surface of the cornea, there is an iris consisting of a flat disk that tangentially extends along an entire internal perimeter of the cornea; below the iris and in contact with its posterior wall, there is a cataract, surrounded anteriorly and posteriorly by two circular sheets that characterize their respective anterior and posterior capsules, peripherally delimited by a ring; below, there is a cylindrical structure; in sequence and in coupling, there is a cylinder that is fixed by its posterior portion to a larger posterior cut of a spherical structure, which also has a smaller anterior cut, through which the cornea is presented; the spherical structure rests on the edges of a hollow cylinder and is sheltered at a top in its largest diameter by internal edges of a second hollow cylinder, which partially covers the spherical structure.

2. THE ARRANGEMENT APPLIED TO A SIMULATOR FOR SURGICAL TRAINING IN OPHTHALMOLOGY according to claim 1, wherein the cataract is a biconvex or plano-convex lens.

3. THE ARRANGEMENT APPLIED TO A SIMULATOR FOR SURGICAL TRAINING IN OPHTHALMOLOGY according to claim 1, wherein the spherical structure has the potential for rotational movement.

4. AN ARRANGEMENT APPLIED TO A SIMULATOR FOR SURGICAL TRAINING IN OPHTHALMOLOGY, comprising at least one cornea, one iris, one sclera, one cylinder and an internal channel that surrounds an internal part of the simulator.

5. THE ARRANGEMENT APPLIED TO A SIMULATOR FOR SURGICAL TRAINING IN OPHTHALMOLOGY according to claim 4, wherein the internal channel is a closed channel, with a substantially tubular shape.

6. THE ARRANGEMENT APPLIED TO A SIMULATOR FOR SURGICAL TRAINING IN OPHTHALMOLOGY according to claim 4, wherein the internal channel is positioned in the sclera, in a location close to the cornea and the iris; in the cornea, in a location close to the sclera and the iris; or at a junction site between the cornea and the sclera.

7. AN ARTIFICIAL CATARACT, comprising a plurality of grooves arranged uniformly spaced around the cataract.

8. THE ARTIFICIAL CATARACT according to claim 7, wherein the grooves extend from an anterior part of the cataract (50) until they meet in a convex posterior part of the cataract.

9. THE ARTIFICIAL CATARACT according to claim 7, wherein, the grooves extend only partially inwardly the upper part of the cataract.
